(19) United States
(12) Patent Application Publication
(10) Pub. No.: US 2011/0178414 A1
(43) Pub. Date: Jul. 21, 2011

(54) GIOLOGICAL INFORMATION DETECTOR AND BIOLOGICAL INFORMATION MEASURING DEVICE

(75) Inventors: Yoshitaka IIJIMA, Shiojiri (JP); Hideto YAMASHITA, Suwa (JP); Hideo MIYASAKA, Okaya (JP)

(73) Assignee: SEIKO EPSON CORPORATION, Tokyo (JP)

(21) Appl. No.: 12/982,439

(22) Filed: Dec. 30, 2010

(30) Foreign Application Priority Data
Jan. 21, 2010 (JP) 2010-010721

(51) Int. Cl. A61B 6/00; H01J 40/14

(52) U.S. Cl. 600/479; 250/216

(57) ABSTRACT

A biological information detector includes a light-emitting part, a light-receiving part, a reflecting part, and a substrate. The light-receiving part is for receiving light having biological information, where the light is emitted by the light-emitting part and reflected at a detection site of a test subject. The reflecting part is for reflecting the light emitted by the light-emitting part or the light having biological information. The substrate has a first surface and a second surface facing the first surface, formed from a material that is transparent with respect to a wavelength of the light emitted by the light-emitting part, at least one of the first surface and the second surface of the substrate has a light-blocking region containing wiring leading to at least the other of the light-emitting part and the light-receiving part.

### CROSS-REFERENCE TO RELATED APPLICATIONS

This application claims priority to Japanese Patent Application No. 2010-010721 filed on Jan. 21, 2010. The entire disclosure of Japanese Patent Application No. 2010-010721 is hereby incorporated herein by reference.

### BACKGROUND

1. Technological Field

The present invention relates to a biological information detector and a biological information measuring device and similar devices.

2. Background Technology

A biological information measuring device measures human biological information such as, for example, pulse rate, blood oxygen saturation level, body temperature, or heart rate; and an example of a biological information measuring device is a pulse rate monitor for measuring the pulse rate. Also, a biological information measuring device such as a pulse rate monitor may be installed in a clock, a mobile phone, a pager, a PC, or another electrical device, or may be combined with the electrical device. The biological information measuring device has a biological information detector for detecting biological information, and the biological information detector includes a light-emitting part for emitting light towards a detection site of a test subject (e.g., a user), and a light-receiving part for receiving light having biological information from the detection site.

In Patent Citation 1, there is disclosed a pulse rate monitor (or in a broader sense, a biological information measuring device). A light-receiving part (e.g., a light-receiving part 12 in FIG. 16 of Patent Citation 1) of the pulse rate monitor receives light reflected at a detection site (e.g., dotted line in FIG. 16 of Patent Citation 1) via a diffusion reflection plane (e.g., reflecting part 131 in FIG. 16 of Patent Citation 1). In an optical probe 1 in Patent Citation 1, a light-emitting part 11 and the light-receiving part 12 overlap with respect to the plan view, and the size of the optical probe is reduced.

Related Art

JP-A 2004-337605 (hereinafter Patent Citation 1) is an example of the related art.

### SUMMARY

### Problems to Be Solved by the Invention

According to paragraph [0048] in Patent Citation 1, a substrate 15 is formed so that a side facing an inner side of the reflecting part 131 is a diffuse reflecting surface. Specifically, the substrate 15 according to Patent Citation 1 blocks light emitted by the light-emitting part 11, and the entirety of the substrate 15 forms a light-blocking region. Therefore, the detection accuracy of the biological information detector is poor.

According to several aspects of the invention, it is possible to provide a biological information detector and a biological information measuring device in which the detection accuracy or the measurement accuracy can be improved.

### Means Used to Solve the Above-Mentioned Problems

A first aspect of the invention relates to a biological information detector, characterized in including:

a light-emitting part;

a light-receiving part for receiving light having biological information, the light being light emitted by the light-emitting part and reflected at a detection site of a test subject;

a reflecting part for reflecting the light emitted by the light-emitting part or the light having biological information; and

a substrate having a first surface and a second surface facing the first surface, the light-receiving part being positioned on one of either the first surface or the second surface, and the light-emitting part being positioned on another of either the first surface or the second surface; wherein

the substrate is formed from a material that is transparent with respect to a wavelength of the light emitted by the light-emitting part; and

at least one of either the first surface or the second surface of the substrate has a light-blocking region containing wiring leading to at least one of either the light-emitting part or the light-receiving part, and a light transmission film that is transparent with respect to the wavelength of the light emitted by the light-emitting part, the light transmission film being positioned, with respect to the plan view, at least on a region on the substrate excluding the light-blocking region.

According to the first aspect of the invention, the light from the light-emitting part is reflected at the detection site and turned into the light containing biological information, and the light containing the biological information is detected at the light-receiving part, whereby the biological information is detected. The light from the light-emitting part may be reflected at the reflecting part and directed at the detection site, or, alternatively, the light containing biological information from the detection site may be reflected at the reflecting part and detected at the light-receiving part. In either instance, the light emitted by the light-emitting part or the light having the biological information is capable of transmitting through the region excluding the light-blocking region containing the wiring to at least one of either light-emitting part or the light-receiving part. Therefore, the amount of light reaching the light-receiving part or the detection site increases, and the detection accuracy of the biological information detector improves. Also, in the region excluding the light-blocking region with respect to the plan view, having the substrate covered by the light transmission film, at a minimum, makes it possible to fill over and minimize roughness on at least one surface of the substrate with the light transmission film and reduce dispersion of light on the rough surface. Specifically, the light transmission film is capable of smoothening at least one surface of the substrate and improving the transmittance of light travelling in a straight line. This is particularly effective in an instance in which the substrate surface is deliberately formed as a rough surface in order to prevent the wiring or another component from peeling off. Therefore, the amount of light reaching the light-receiving part or the detection site increases, and the detection accuracy of the biological information detector improves further. The light transmission film may be positioned at least on the region on the substrate excluding the light-blocking region with respect to the plan view, and may also be formed on a region that overlaps the light-blocking region with respect to the plan view.

According to a second aspect of the invention, the wiring may have a pad for providing a connection to the light-receiving part, the connecting pad being on the one of either the first surface or the second surface;

the substrate may have an opening part provided, as viewed from above, adjacent to the connecting pad on the one of either the first surface or the second surface, the light transmission film not being positioned in the opening part; and

the opening part may, with respect to the plan view, overlap with the light-blocking region on the other of either the first surface or the second surface of the substrate.

Thus, the substrate in a vicinity of the connecting pad for connecting to the light-receiving part may have the opening part instead of the light transmission film. The connecting pad for connecting to the light-receiving part must be exposed so that wire bonding or another bonding is possible, and cannot be entirely covered by the light transmission film. In at least one of the connecting pad or the light transmission film, an allowance is made for the opening part to be formed as a result of positional displacement being created by an error during a photolithography process or another manufacturing process. However, in an instance in which the substrate has the opening part on, e.g., the first surface, the light-blocking region of the substrate is present on the second surface opposite the opening part. In a region overlapping the light-blocking region with respect to the plan view, even in the presence of the opening part, light does not pass through the opening part. In contrast, in an instance in which the opening part does not overlap the light-blocking region with respect to the plan view, the light emitted by the light-emitting part or the light having the biological information disperse at the opening part of the substrate.

According to a third aspect of the invention, the wiring may have a pad for providing a connection to the light-emitting part, the connecting pad being on the other of either the first surface or the second surface;

the substrate may have an opening part provided, as viewed from above, adjacent to the connecting pad on the other of either the first surface of the second surface, the light transmission film not being positioned in the opening part; and

the opening part may, with respect to the plan view, overlap with the light-blocking region on the one of either the first surface or the second surface of the substrate.

Thus, the substrate in a vicinity of the connecting pad for connecting to the light-emitting part may have the opening part instead of the light transmission film. The connecting pad for connecting to the light-emitting part must be exposed so that wire bonding or another bonding is possible, and cannot be entirely covered by the light transmission film. In at least one of the connecting pad or the light transmission film, an allowance is made for the opening part to be formed as a result of positional displacement being created by an error during a photolithography process or another manufacturing process. However, again, in an instance in which the substrate has the opening part on e.g., the second surface, the light-blocking region of the substrate is present on the first surface opposite the opening part.

According to a fourth aspect of the invention, the biological infatuation detector may have a false wiring positioned on the light-blocking region overlapping the opening part with respect to the plan view, the light-blocking region being on the other of either the first surface or the second surface of the substrate.

In an instance in which the substrate has the opening part on e.g., the first surface, the false wiring may be present on the second surface facing the opening part. It is thus possible to readily form the light-blocking region using the false wiring.

According to a fifth aspect of the invention, the wiring may have a connecting part in contact with an electrode of the light-receiving part, and the connecting part may be positioned on the light-blocking region overlapping the opening part with respect to the plan view, the light-blocking region being on the one of either the first surface or the second surface of the substrate.

In an instance in which the substrate has the opening part on e.g., the second surface, the connecting part (i.e., wiring) in contact with the electrode of the light-receiving part may be present on the first surface corresponding to the opening part. The light-blocking region may be readily formed by extending the connecting part (i.e., the wiring).

According to a sixth aspect of the invention,

the connecting pad may have an exposed part in which a part of a surface of the connecting pad is exposed,

the opening part may be adjacent to the exposed part with respect to the plan view, and

another part of the surface of the connecting pad may be covered by the light transmission film.

Providing the light transmission film so as to overlap the other part of the surface of the connecting pad thus eliminates a gap (i.e., the opening) in this region. Meanwhile, to account for an error during manufacturing of the light transmission film or another component, an opening may be formed between the exposed part, which is a part of the surface of the connecting part and which cannot be covered by the light transmission film, and the light transmission film. The opening must overlap the light-blocking region with respect to the plan view.

According to a seventh aspect of the invention, the wiring may also have a pad for providing a connecting to at least one of the light-emitting part or the light-receiving part,

the connecting pad may have an exposed part in which a part of a surface of the connecting pad is exposed, and

a periphery of the surface of the connecting pad may be covered by the light transmission film.

The connecting pad for connecting to the light-emitting part or the light-receiving part must be exposed so that wire bonding or another type of bonding is possible, and cannot be entirely covered by the light transmission film. In at least one of the connecting pad or the light transmission film, although an error during a photolithography process or another manufacturing process causes a positional displacement, even in an instance in which a maximum positional displacement is generated, the periphery of the exposed part of the connecting pad is covered by the light transmission film, and the opening part is prevented from forming in a region where the opening part is not necessary.

An eighth aspect of the invention relates to a biological information measuring device characterized in including:

the biological information detector described above; and

a biological information measuring part for measuring the biological information from a light reception signal generated in the light-receiving part; wherein

the biological information is a pulse rate.

According to the eighth aspect of the invention, the biological information detector whose detection accuracy has been improved can be used to improve the measurement accuracy of the biological information measuring device.

### BRIEF DESCRIPTION OF THE DRAWINGS

FIGS. 1(A) and 1(B) are examples of a biological information detector according to a present embodiment;

FIGS. 2(A), 2(B), and 2(C) are schematic diagrams showing an irradiation region in which light emitted by a light-emitting part or light having biological information travels to a substrate;

FIGS. 3(A) and 3(B) are an example of a layout of a light transmission film and wiring;

FIGS. 4(A), 4(B), 4(C), and 4(D) are schematic diagrams showing the rationale for forming an opening part and a principle behind preventing the opening part from being formed;

FIGS. 5(A) and 5(B) are examples of a layout of the light transmission film;

FIGS. 6(A) and 6(B) are examples of a layout surrounding a connecting pad;

FIG. 7 is another example of a layout of the light transmission film and the wiring;

FIGS. 8(A) and 8(B) are other examples of a layout surrounding the connecting pad;

FIG. 9 is an example of intensity characteristics of light emitted by the light-emitting part;

FIG. 10 is an example of transmission characteristics of light passing through the substrate coated with the light transmission film;

FIG. 11 is another example of the biological information detector according to the present embodiment;

FIG. 12 is another example of a layout surrounding the connecting pad;

FIGS. 13(A) and 13(B) is an example of the outer appearance of a biological information measuring device containing the biological information detector; and

FIG. 14 is an example of a configuration of the biological information measuring device.

### DETAILED DESCRIPTION OF EXEMPLARY EMBODIMENTS

A description shall now be given for the present embodiment. The present embodiment described below is not intended to unduly limit the scope of the Claims of the present embodiment. Not every configuration described in the present embodiment is necessarily an indispensible constituent feature of the invention.

### 1. Biological Information Detector

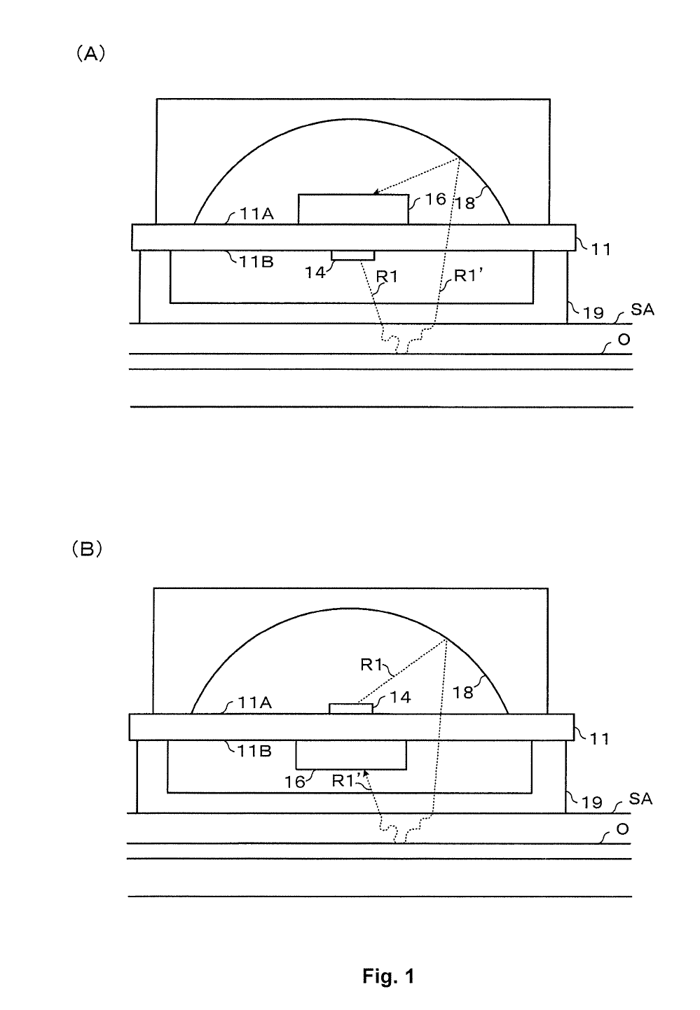

FIGS. 1(A) and 1(B) show an example of respective configurations of the biological information detector according to the present embodiment. As shown in FIGS. 1(A) and 1(B), the biological information detector includes a substrate 11, a light-emitting part 14, a light-receiving part 16, and a reflecting part 18. Also, although not shown in FIGS. 1(A) and 1(B), the biological information detector includes a wiring and a light transmission film as described further below. Also, as shown in FIGS. 1(A) and 1(B), the biological information detector may include a protecting part 19.

As shown in FIGS. 1(A) and 1(B), the light-emitting part 14 emits a light R1 directed at a detection site O of a test subject (e.g., a user). The light-receiving part 16 receives a light R1′ having biological information (i.e., reflected light), the light R1′ produced by the light R1 emitted by the light-emitting part 14 being reflected at the detection site O. The reflecting part 18 reflects the light R1 emitted by the light-emitting part 14 or the light R1′ having the biological information (i.e., the reflected light). The reflecting part 18 may have a reflecting surface on a dome surface (i.e., a spherical surface or a parabolic surface) provided on a light path between the light-emitting part 14 and the light-receiving part 16. The substrate 11 may have a first surface (e.g., a front surface) 11A and a second surface (e.g., a reverse surface) 11B that is opposite the first surface 11A. The light-receiving part 16 is positioned on one of either the first surface 11A or the second surface 11B (the first surface 11A in FIG. 1(A) and the second surface 11B in FIG. 1(B)). The light-emitting part 14 is positioned on another of either the first surface 11A or the second surface 11B (the second surface 11B in FIG. 1(A) and the first surface 11A in FIG. 1(B)). The substrate 11 is formed from a material that is transparent with respect to a wavelength of the light R1 emitted by the light-emitting part 14. As described further below, wiring to at least one of the light-emitting part 14 and the light-receiving part 16, and a light transmission film for transmitting the light R1 emitted by the light-emitting part 14, may be formed on the substrate 11. Also, the light transmission film is positioned on at least a region of the substrate 11 excluding, with respect to the plan view, a light-blocking region of the substrate 11 on which the wiring is positioned.

The light R1 emitted by the light-emitting part 14 and the light R1′ having the biological information (i.e., the reflected light) are capable of passing through the substrate 11, which is formed from a transparent material. Therefore, the amount of light reaching the light-receiving part 16 or the detection site O increases, and the detection accuracy of the biological information detector improves. Also, the substrate 11 is covered with the light transmission film, thereby making it possible to fill in and smoothen roughness on at least one surface of the substrate 11, and to reduce dispersion of light on the rough surface. Specifically, the light transmission film is capable of smoothening at least surface of the substrate 11 and improving the transmittance of light travelling in a straight line. Therefore, the amount of light reaching the light-receiving part 16 or the detection site O increases, and the detection accuracy of the biological information detector improves further.

According to paragraph [0048] of Patent Citation 1, the substrate 15 is formed so that a side facing an inner side of the reflecting part 131 is a diffuse reflecting surface. Specifically, the substrate 15 according to Patent Citation 1 is not required to be formed from a transparent material, the substrate 15 according to Patent Citation 1 blocks light emitted by the light-emitting part 11, and as a result, the entirety of the substrate 15 forms a light-blocking region. Therefore, the detection accuracy of the biological information detector is poor.

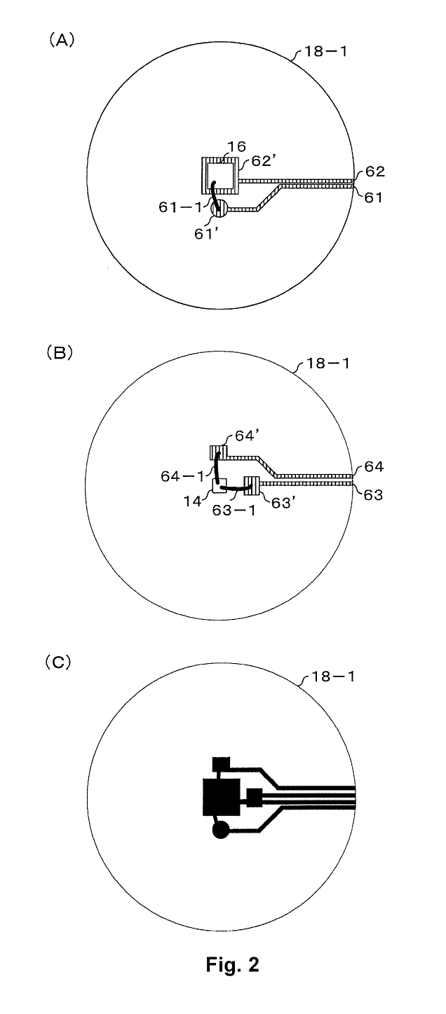

FIGS. 2(A), 2(B), and 2(C) are schematic diagrams showing an irradiation region in which light R1 emitted by the light-emitting part 14 or the light R1′ having biological information (i.e., the reflected light) travels to the substrate 11. The irradiation region may be defined, for example, by a boundary 18-1 between the reflecting surface of the reflecting part 18 (i.e., the dome surface in each of the examples shown in FIGS. 1(A) and 1(B)) and the substrate 11. The boundary 18-1 has, for example, a circular profile.

As shown in FIG. 2(A), in e.g., plan view when viewed from a side of the light-receiving part 16 in FIG. 1(A), a wiring 61 for connecting to an anode (or in a broader sense, an electrode) of the light-receiving part 16 is formed on the first surface 11A of the substrate 11. A wiring 62 that connects to a cathode (or in a broader sense, an electrode) of the light-receiving part 16 is also formed on the first surface 11A of the substrate 11. In the example shown in FIG. 2(A), the wiring 61 has a connecting pad 61′ that connects to the light-receiving part 16, and a bonding wire 61-1. The connecting pad 61′ of the wiring 61 is connected to the anode of the light-receiving part 16 via the bonding wire 61-1. In the example shown in FIG. 2(A), the wiring 62 has a connecting part 62′ in contact with the cathode of the light-receiving part 16, and the connecting part 62′ of the wiring 62 is directly connected to the cathode of the light-receiving part 16 via e.g., an adhesive (not shown). An example of an electroconductive adhesive that may be used is silver paste. In the example shown in FIG. 1(B), the wiring 61, 62 and similar components are formed on the second surface 11B of the substrate 11.

As shown in FIG. 2(B), with respect to plan view when viewed, e.g., from a side of the light-emitting part 14 in FIG. 1(A), a wiring 63 for connecting to a cathode of the light-emitting part 14 is formed on the second surface 11 B of the substrate 11. A wiring 64 for connecting to an anode of the light-emitting part 14 is also formed on the second surface 11B of the substrate 11. In the example shown in FIG. 2(B), the wiring 63 has a connecting pad 63′ that connects to the light-receiving part 14, and a bonding wire 63-1. The connecting pad 63′ of the wiring 63 is connected to the cathode of the light-receiving part 16 via the bonding wire 63-1. In the example shown in FIG. 2(B), the wiring 64 has a connecting part 64′ that connects to the light-receiving part 14, and a bonding wire 64-1. The connecting pad 64′ of the wiring 64 is connected to the anode of the light-receiving part 14 via the bonding wire 64-1. In the example shown in FIG. 1(B), the wiring 63, 64 and similar components are formed on the first surface 11A of the substrate 11.

The configuration of the wiring 63 and the wiring 64 to the light-emitting part 14 and the wiring 61 and the wiring 62 to the light-receiving part 16 is not limited by the examples shown in FIGS. 2(A) and 2(B). For example, the shape of the connecting pad 61′ of the wiring 61 may, instead of being circular as shown in FIG. 2(A), be, e.g., square, elliptical, polygonal, or describing another shape. The shape of the connecting pad 63′ of the wiring 63 may, instead of being rectangle as shown in FIG. 2(B), be, e.g., circular, elliptical, polygonal, or describing another shape. Also, although in the example shown in FIG. 2(A), the light-receiving part 16 has the cathode on a bottom surface, the light-receiving part 16 may have the cathode on a front surface in a similar manner to the anode.

As shown, for example, in FIG. 1(A), in an instance in which the light R1′ having the biological information (i.e., the reflected light) is directed to the substrate 11, the light R1′ having the biological information (i.e., the reflected light) reaches the irradiation region defined by the boundary 18-1 between the reflecting surface of the reflecting part 18 and the substrate 11.

In an instance in which the wiring 63 and the wiring 64 to the light-emitting part 14 are present as shown in FIG. 2(B), at least the wiring 63 and the wiring 64 block or reflect the light R1′ having the biological information (i.e., the reflected light) and form a light-blocking region. Specifically, of the irradiation region, the light-blocking region deters the light R1′ having the biological information (i.e., the reflected light) from entering the substrate 11. Also, even in an instance where the light R1′ having the biological information (i.e., the reflected light) enters an interior of the substrate 11, in an instance where the wiring 61 and the wiring 62 to the light-receiving part 16 are present as shown in FIG. 2(A), at least the wiring 61 and the wiring 62 deter the light R1′ having the biological information (i.e., the reflected light) from leaving the interior towards an exterior of the substrate 11. The light-blocking region of the substrate 11, where the wiring 61, the wiring 62, the wiring 63, and the wiring 64 are positioned, thus deter the light R1′ having the biological information (i.e., the reflected light) from reaching the reflecting part 18. Specifically, the light R1′ having the biological information (i.e., the reflected light) is capable of transmitting through a region of the substrate 11 excluding the light-blocking region of the substrate 11.

As shown, for example, in FIG. 1(B), in an instance in which the light R1 emitted by the light-emitting part 14 is travelling to the substrate 11, the light R1 emitted by the light-emitting part 14 reaches the irradiation region of the substrate 11. In an instance in which the wiring 61 and the wiring 64 to the light-emitting part 14 are present as shown in FIG. 2(A), at least the wiring 61 and the wiring 62 block or reflect the light R1 emitted by the light-emitting part 14 and form a light-blocking region. Specifically, of the irradiation region, the light-blocking region deters the light R1 emitted by the light-emitting part 14 from entering the substrate 11. Also, even in an instance where the light R1 emitted by the light-emitting part 14 enters an interior of the substrate 11, in an instance where the wiring 63 and the wiring 64 to the light-receiving part 14 are present as shown in FIG. 2(B), at least the wiring 63 and the wiring 64 deter the light R1 emitted by the light-emitting part 14 from leaving the interior towards an exterior of the substrate 11. The light-blocking region of the substrate 11, where the wiring 61, the wiring 62, the wiring 63, and the wiring 64 are positioned, thus deter the light R1 emitted by the light-emitting part 14 from reaching the detection site O.

FIG. 2(C) shows a light-blocking region within the irradiation region as shown in plan view. The light-blocking region is shown in black in the example shown in FIG. 2(C). As shown in FIG. 2(C), the light-blocking region can be defined, with respect to the plan view, by the wiring 61 (including the connecting pad 61′ and the bonding wire 61-1) and the wiring 62 (including the connecting part 62′) shown in FIG. 2(A), and the wiring 63 (including the connecting pad 63′ and the bonding wire 63-1) and the wiring 64 (including the connecting pad 64′ and the bonding wire 64-1) shown in FIG. 2(B).

The light transmission film may be positioned on a region of the substrate 11 excluding, with respect to the plan view, the light-blocking region of the substrate 11 where the wiring 61, the wiring 62, the wiring 63, and the wiring 64 are positioned. The light transmission film may be formed on the first surface 11A only, formed on the second surface 11B only, or formed on both of the first surface 11A and the second surface 11B. For example, in the example shown in FIG. 2(A), the light transmission film may be formed within the irradiation region excluding the wiring 61, the connecting pad 61′, the wiring 62, and the connecting part 62′. In the example shown in FIG. 2(B), the light transmission film may be formed within the irradiation region excluding the wiring 63, the connecting pad 63′, the wiring 64, and the connecting pad 64′.

The first surface 11A and the second surface 11B of the substrate 11 may be manufactured or processed so as to form a rough surface so that the wiring 61, the wiring 62, the wiring 63, and the wiring 64 on the substrate 11 do not peel off Specifically, the entirety of the first surface 11A and the second surface 11B of the substrate 11, including a surface on which the wiring 61, the wiring 62, the wiring 63, and the wiring 64 are formed, are formed as a rough surface. The rough surface is useful in terms of reducing the likelihood of the wiring 61 and the other wirings peeling away. However, in terms of being a light-transmissive surface, the rough surface causes dispersion and is not preferable. Therefore, the light transmission film is formed on at least one of the first surface 11A and the second surface 11B, whereby the roughness on at least one surface of the substrate 11 is filled with the light transmission film, and the smoothness of a light-transmitting region of the substrate 11 other than the light-blocking region is improved. Specifically, the light transmission film 11-1 on the substrate 11 is a smoothening film, and can therefore reduce dispersion of light on the rough surface of the substrate 11 during transmission of the light through the substrate 11. Specifically, the presence of the light transmission film smoothens at least one surface of the substrate 11 and improves transmittance of light travelling in a straight line. Therefore, the amount of light reaching the light-receiving part 16 or the detection site O increases, and the detection accuracy of the biological information detector is increased.

Also, as shown in FIGS. 1(A) and 1(B), the biological information detector may also include a protecting part 19. The protecting part 19 protects the light-emitting part 14 or the light-receiving part 16. In the example shown in FIG. 1(A), the protecting part 19 protects the light-emitting part 14. In the example shown in FIG. 1(B), the protecting part 19 protects the light-receiving part 16. The substrate 11 held between the reflecting part 18 and the protecting part 19, the light-emitting part 14 is positioned on the substrate 11 on one of either a side towards the reflecting part 18 or a side towards the protecting part 19, and the light-receiving part 16 is positioned on the substrate 11 on another of either the side towards the reflecting part 18 or the side towards the protecting part 19. In the example shown in FIG. 1(A), the light-receiving part 16 is placed on the substrate 11 on the side towards the reflecting part 18 (or specifically, the first surface 11A of the substrate 11) and the light-emitting part 14 is placed on the substrate 11 on the side towards the protecting part 19 (or specifically, the second surface 11B of the substrate 11). In the example shown in FIG. 1(B), the light-emitting part 14 is placed on the substrate 11 on the side towards the reflecting part 18 (i.e., the first surface) and the light-receiving part 16 is placed on the substrate 11 on the side towards the protecting part 19 (i.e., the second surface). The protecting part 19 has a surface in contact with the test subject, and the protecting part 19 is formed from a material that is transparent with respect to the wavelength of the light R1 emitted by the light-emitting part 14 (e.g., glass). The substrate 11 is also formed from a material that is transparent with respect to the wavelength of the light R1 emitted by the light-emitting part 14 (e.g., polyimide).

Since the substrate 11 is held between the reflecting part 18 and the protecting part 19, even when the light-emitting part 14 and the light-receiving part 16 are positioned on the substrate 11, there is no need to separately provide a mechanism for supporting the substrate 11 itself, and the number of components is smaller. Also, since the substrate 11 is formed from a material that is transparent with respect to the emission frequency, the substrate 11 can be disposed on a light path from the light-emitting part 14 to the light-receiving part 16, and there is no need to accommodate the substrate 11 at a position away from the light path, such as within the reflecting part 18. A biological information detector that can be readily assembled can thus be provided. Also, the reflecting part 18 is capable of increasing the amount of light reaching the light-receiving part 16 or the detection site O, and the detection accuracy (i.e., the signal-to-noise ratio) of the biological information detector increases.

In Patent Citation 1, it is necessary to install the light-emitting part 11, the light-receiving part 12, the substrate 15, and the transparent material 142 in the interior of the reflecting part 131. Therefore, a small optical probe 1 cannot be assembled with ease.

In the example shown in FIGS. 1(A) and 1(B), the detection site O (e.g., a blood vessel) is within the test subject. The first light R1 travels into the test subject and diffuses or scatters at the epidermis, the dermis, and the subcutaneous tissue. The first light R1 then reaches the detection site O, and is reflected at the detection site O. The reflected light R1′ reflected at the detection site O diffuses or scatters at the subcutaneous tissue, the dermis, and the epidermis. In FIG. 1(A), the reflected light R1′ travels to the reflecting part 18. In FIG. 1(B), the first light R1 travels to the detection site O via the reflecting part 18. The first light R1 is partially absorbed at the detection site O (i.e., the blood vessel). Therefore, due to an effect of a pulse, the rate of absorption at the blood vessel varies, and the amount of the reflected light R1′ reflected at the detection site O also varies. Biological information (e.g., pulse rate) is thus reflected in the reflected light R1′ reflected at the detection site O.

In the example shown in FIG. 1(A), the light-emitting part 14 emits the first light R1 towards the detection site O; the reflecting part 18 reflects the reflected light R1′, produced by the first light R1 being reflected at the detection site O, towards the light-receiving part 16; and the light-receiving part 16 receives the reflected light R1′ having the biological information at the detection site O. In the example shown in FIG. 1(B), the light-emitting part 14 emits the first light R1 towards the detection site O via the reflecting part 18, and the light-receiving part 16 receives the reflected light R1′, produced by the first light R1 being reflected, having the biological information at the detection site O.

The thickness of the substrate 11 is, e.g., 10 μm to 1000 μm. Wiring to the light-emitting part 14 and wiring to the light-receiving part 16 may be formed on the substrate 11. The substrate 11 is, e.g., a printed circuit board; however, a printed circuit board is not generally formed from a transparent material, as with the substrate 15 of Patent Citation 1. Specifically, the inventors purposefully used a configuration in which the printed circuit board is formed from a material that is transparent at least with respect to the emission wavelength of the light-emitting part 14. The thickness of the protecting part 19 is, e.g., 1 μm to 1000 μm.

Examples of configurations of the biological information detector are not limited by those shown in FIGS. 1(A) and 1(B), and the shape, or a similar attribute, of a part of the example of configuration (e.g., the light-receiving part 16) may be modified. The biological information may also be blood oxygen saturation level, body temperature, heart rate, or a similar variable; and the detection site O may be positioned at the surface SA of the test subject. In the examples shown in FIGS. 1(A) and 1(B), the first light is shown by a single line; however, in reality, the light-emitting part 14 emits many light beams in a variety of directions.

The light-emitting part 14 is, for example, an LED. The light emitted by the LED has a maximum intensity (or in a broader sense, a peak intensity) within a wavelength range of, e.g., 425 nm to 625 nm, and is, e.g., green in color. The thickness of the light-emitting part 14 is, e.g., 20 μm to 1000 μm. The light-receiving part 16 is, e.g., a photodiode, and can generally be formed by a silicon photodiode. The thickness of the light-receiving part 16 is, e.g., 20 μm to 1000 μm. The silicon photodiode has a maximum sensitivity (or in a broader sense, a peak sensitivity) for received light having a wavelength within a range of, e.g., 800 nm to 1000 nm. Ideally, the light-receiving part 16 is formed by a gallium arsenide phosphide photodiode, and the gallium arsenide phosphide photodiode has a maximum sensitivity (or in a broader sense, a peak sensitivity) for received light having a wavelength within a range of, e.g., 550 nm to 650 nm. Since biological substances (water or hemoglobin) readily allow transmission of infrared light within a wavelength range of 700 nm to 1100 nm, the light-receiving part 16 formed by the gallium arsenide phosphide photodiode is more capable of reducing noise components arising from external light than the light-receiving part 16 formed by the silicon photodiode.

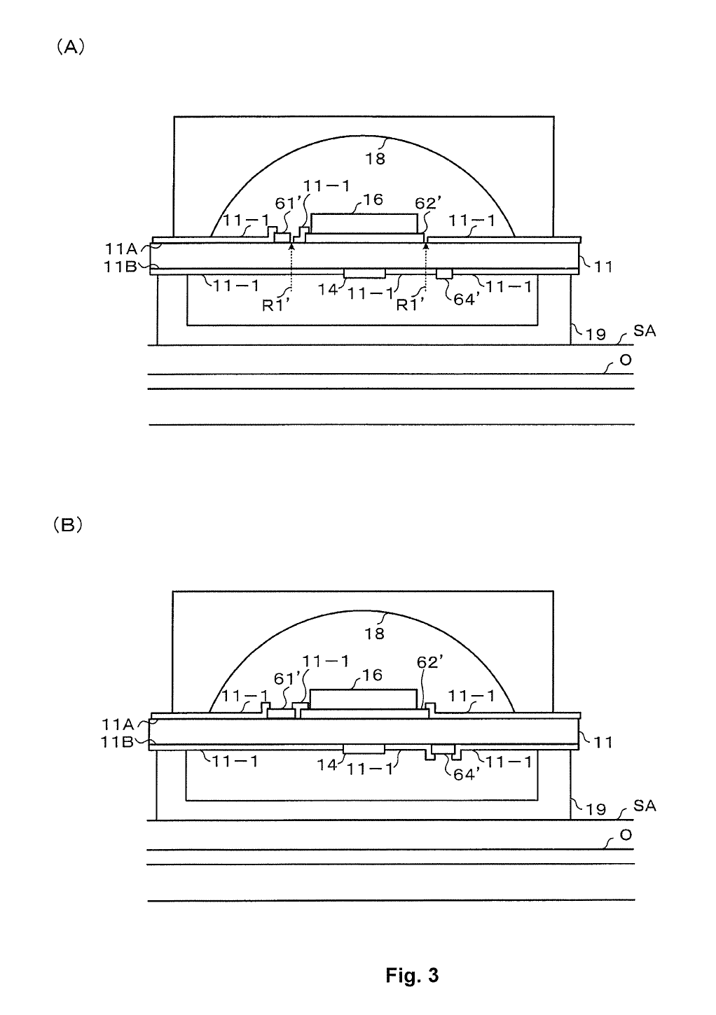

FIGS. 3(A) and 3(B) show examples of a layout of the light transmission film and the wiring. Structures that are identical to those in the example described above are affixed with the same numerals, and a description of the structures is not provided. Although FIGS. 3(A) and 3(B) correspond to FIG. 1(A), the light transmission film and the wiring can also be positioned in the example of configuration shown in FIG. 1(B). A description will now be given for FIGS. 3(A) and 3(B) corresponding to FIG. 1(A). The light transmission film 11-1 may be formed from, e.g., a solder resist (or in a broader sense, a resist). The refraction index of the light transmission film 11-1 is preferably between the refraction index of air and the refraction index of the substrate 11. Also, the refraction index of the light transmission film 11-1 is preferably closer to the refraction index of the substrate 11 than the refraction index of air. In such an instance, it is possible to reduce reflection of light at an interface between the substrate 11 and the light transmission film 11-1 or the interface between the light transmission film 11-1 and air.

As shown in FIG. 3(A), the light transmission film 11-1 and the connecting pad 64′, as well as the light-emitting part 14, are positioned on the second surface 11B of the substrate 11. Although not shown in FIG. 3(A), the wiring 64, the connecting pad 63′, and the wiring 63 are also positioned on the second surface of the substrate 11 (see FIG. 2(B)). The light transmission film 11-1 can be positioned on a region of the second surface 11B of the substrate 11 where the wiring 63, the connecting pad 63′, the wiring 64, and the connecting pad 64′ are not positioned.

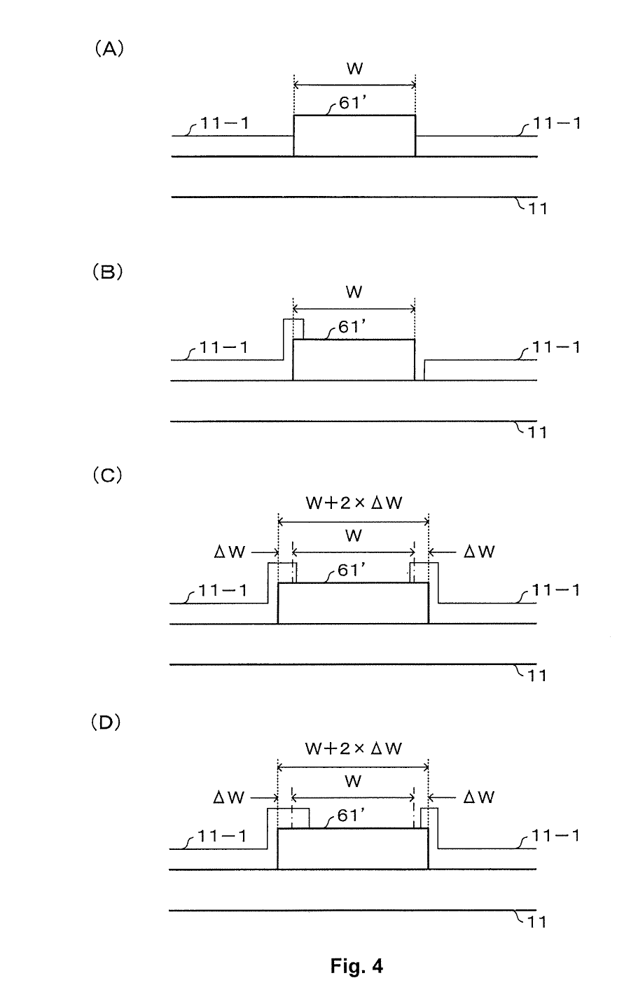

The light transmission film 11-1 can also be positioned on the first surface 11A of the substrate 11, and the light transmission film 11-1 can be positioned on a region of the first surface 11A of the substrate 11 where the wiring 61, the connecting pad 61′, the wiring 62, and the connecting part 62′ are not positioned (see FIG. 2(A)). In the example shown in FIG. 3(A), while the light transmission film 11-1 on the first surface 11A of the substrate 11 is positioned to the right in relation to an intended position (in FIGS. 3(A) and 3(B), the direction of the light-receiving part 16 relative to the connecting pad 61′ is defined as the right), the light transmission film 11-1 on the second surface 11B of the substrate 11 is positioned at an intended position. If the connecting pad 61′ and the light transmission film 11-1 are formed in the intended positions, as shown in FIG. 4(A), no gap is created. However, in FIG. 3(A), e.g., the light transmission film 11-1 is positionally displaced as shown in FIG. 4(B), and a gap δ is thereby created. This is caused by, in an instance in which at least one of either the light transmission film 11-1 or the connecting pad 61′ is formed using, e.g., photolithography, a positional displacement of a photomask or another manufacturing error causing at least one of either the light transmission film 11-1 or the connecting pad 61′ to not be positioned at the intended position. In an instance in which the gap δ shown in FIG. 4(B) has been created between the connecting pad 61′ and the light transmission film 11-1, in the example shown in FIG. 3(A), when the light R1′ having the biological information (i.e., the reflected light) leaves the interior of the substrate 11 towards the exterior, the presence of a gap δ as described above thus causes the light R1′ having the biological information (i.e., the reflected light) to disperse at the rough surface of the first surface 11A of the substrate 11.

In the example shown in FIG. 3(B), the light transmission film 11-1 on the first surface 11A of the substrate 11 is positioned to the right of an intended position, while the light transmission film 11-1 on the second surface 11B of the substrate 11 is positioned at an intended position. However, in cross-sectional view, the size of the area of the connecting pad 61′ shown in FIG. 3(B) is larger than that of the connecting pad 61′ shown in FIG. 1(A), accounting for an error during manufacture of the light transmission film 11-1 which is subsequently formed. Specifically, the size of the connecting pad 61′ in FIG. 3(B) can be increased in accordance with a maximum positional displacement of the light transmission film 11-1. As shown in FIG. 4(C), W is used to represent an inherent size of the connecting pad 61′ in FIG. 3(A), and ΔW is used to represent the maximum amount by which the light transmission film 11-1 is displaced in one direction. The one direction in which the light transmission film 11-1 undergoes displacement refers to at least one of orthogonal axes x, y on a two-dimensional plane on which the substrate 11 is scanned e.g., during exposure. Since the light transmission film 11-1 is present on both the left and right of the connecting pad 61′, the size of the connecting pad 61′ can be set to W+2×ΔW, as shown in FIG. 4(C) in turn from FIG. 4(A). In a state shown in FIG. 4(C), in which the connecting pad 61′ and the light transmission film 11-1 are formed at intended positions, a mask is configured to the light transmission film 11-1 on both sides so that each of the light transmission films 11-1 overlaps the connecting pad 61′ by a length equal to or larger than ΔW. According to the configuration described above, even in an instance in which, for example, the light transmission film 11-1 is positionally displaced to the right by the maximum amount ΔW as shown in the example in FIG. 3(B), both ends of the wiring connecting pad 61′ are overlapped by the light transmission film 11-1 as shown in FIG. 4(D), and the gap δ shown in the example in FIG. 4(B) can be minimized. Also, even in an instance in which the light transmission film 11-1 on the second surface 11B of the substrate 11 is not positioned at an intended position, a gap of such description can be minimized. Also, when ΔW/2 is defined as a maximum amount by which each of the respective light transmission films 11-1 and the connecting pads 61′, 64′ on each of the first surface 11A and the second surface 11B of the substrate 11 can be displaced in one direction, even in an instance in which displacement takes place by a maximum amount of ΔW/2 in mutually opposing directions (i.e., resulting in a relative displacement of ΔW), setting a mask as shown in FIG. 4(C) makes it possible to inhibit the gap δ from being created.

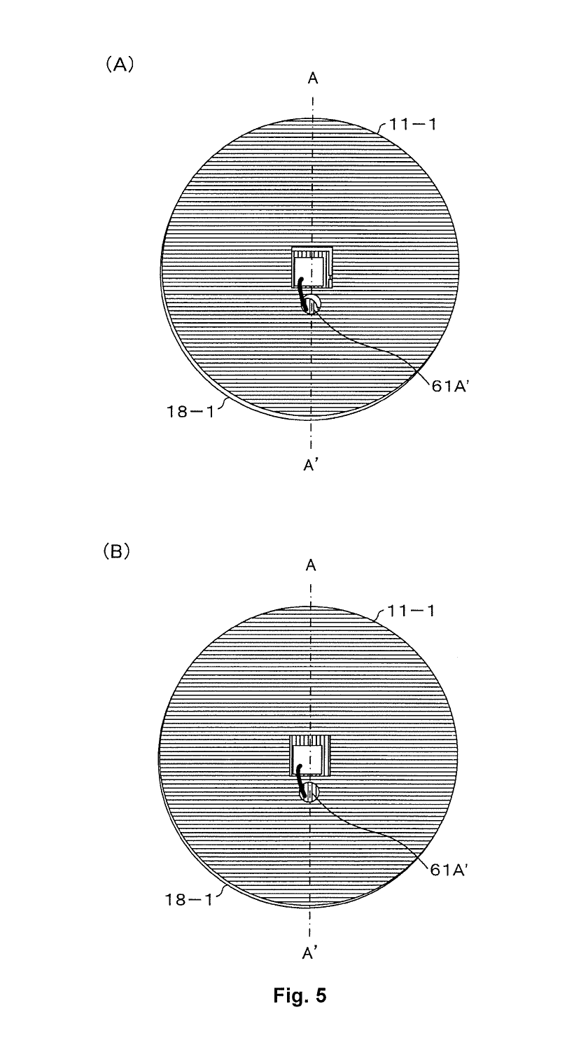

FIGS. 5(A) and 5(B) each show an example of a configuration of the light transmission film 11-1. Both of FIGS. 5(A) and 5(B) correspond to FIG. 2(A). A cross-sectional view along the line A-A′ in FIG. 5(A) corresponds to FIG. 3(A), and a cross-sectional view along the line A-A′ in FIG. 5(B) corresponds to FIG. 3(B). Only a region of the light transmission film 11-1 on the first surface of the substrate 11 that corresponds to the boundary 18-1 between the reflecting surface of the reflecting part 18 and the substrate 11 is shown in FIGS. 5(A) and 5(B). The light transmission film 11-1 may be formed between the first surface 11A of the substrate 11 and the reflecting part 18, as shown in FIGS. 3(A) and 3(B). In FIGS. 5(A) and 5(B), the light transmission film 11-1 on the first surface 11A of the substrate 11 is positioned upward of an intended position (in FIGS. 5(A) and 5(B), label A is defined as an upward direction and label A′ is defined as a downward direction). Also, as shown in FIGS. 5(A) and 5(B), the light transmission film 11-1 on the first surface of the substrate 11 may cover a surface of the wiring 61 and a surface of the wiring 62, which are light-blocking regions (see FIG. 2(A)). As shown in FIGS. 5(A) and 5(B), the bonding wire 61-1 is formed on a surface of the connecting pad 61′, and the surface of the connecting pad 61′ cannot entirely be covered by the light transmission film 11-1 (see FIG. 2(A)). Specifically, the connecting pad 61′ has an exposed part 61A′ in which at least a part of the surface of the connecting pad 61′ is exposed (see FIGS. 5(A) and 5(B)).

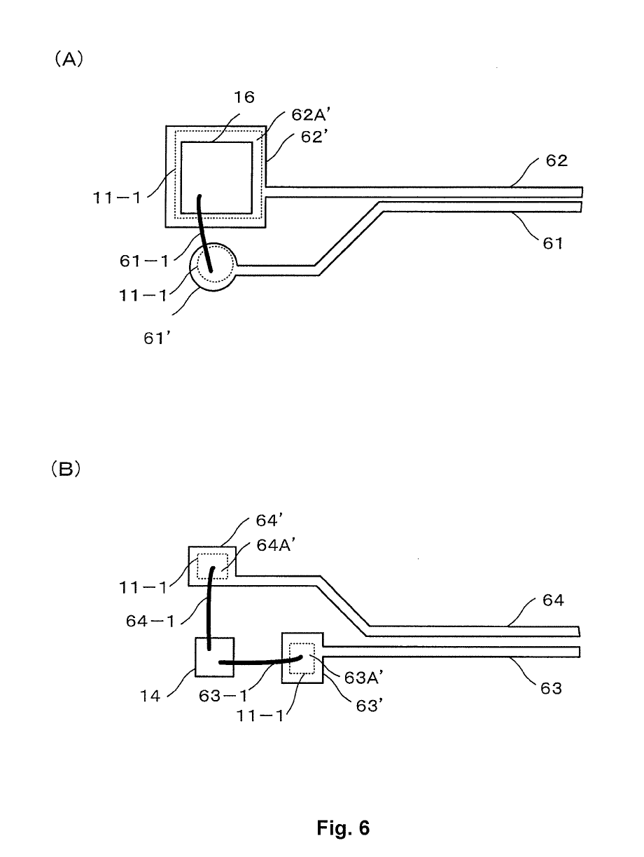

FIGS. 6(A) and 6(B) each show an example of a layout surrounding the connecting pad. FIG. 6(A) shows an example of a layout surrounding the connecting pad 61′ shown in FIG. 3(B). Also, in FIG. 6(A), an edge of the light transmission film 11-1 shown in FIG. 5(B) is shown by a dotted line. As shown in FIG. 6(A), the connecting pad 61′ for connecting to the light-receiving part 16 has the exposed part 61A′ in which at least a part of the surface of the connecting pad 61′ is exposed. The exposed part 61A′ is defined by the edge of the light transmission film 11-1. The bonding wire 61-1 is formed at the exposed part 61A′ of the connecting pad 61′. In the example shown in FIG. 6(A), a periphery of the surface of the connecting pad 61′ is covered by the light transmission film 11-1 which overlaps the connecting pad 61′. Also, in the example shown in FIG. 6(A), the connecting part 62′ for connecting to the light-receiving part 16 has an exposed part 62A′ in which at least a part of a surface of the connecting part 62′ is exposed, and a periphery of the surface of the connecting part 62′ is covered by the light transmission film 11-1 which overlaps the connecting part 62′.

FIG. 6(B) shows an example of a layout surrounding the connecting pad 64′ shown in FIG. 3(B). In the example shown in FIG. 6(B), the connecting pad 64′ for connecting to the light-emitting part 14 has an exposed part 64A′ in which at least a part of a surface of the connecting pad 64′ is exposed, and a periphery of the surface of the connecting pad 64′ is covered by the light transmission film 11-1 which overlaps the connecting pad 64′ (see FIG. 3(B)). Also, in the example shown in FIG. 6(B), as with the connecting pad 64′, the connecting pad 63′ for connecting to the light-emitting part 14 has an exposed part 63A′ in which at least a part of a surface of the connecting pad 63′ is exposed, and a periphery of the surface of the connecting pad 63′ is covered by the light transmission film 11-1 which overlaps the connecting pad 63′. A bonding wire 64-1 and a bonding wire 63-1 are respectively formed on the exposed part 64A′ of the connecting pad 64′ and the exposed part 63A′ of the connecting pad 63′.

Accounting for the error when the light transmission film 11-1 and similar components are manufactured, the connecting pad 61′ and similar components are configured so as to have a larger area than, e.g., a minimum area necessary for wire bonding, and a photomask or another method is used so that the periphery of the surface of the connecting pad 61′ and other connecting pads are covered by the light transmission film 11-1. This makes it possible to eliminate a gap between the light transmission film 11-1 and the periphery of the surface of the connecting pad 61′ and other connecting pads, even in an instance of a mask displacement or another manufacturing error. The light transmission film 11-1 adjacent to the periphery of the surface of the connecting pad 61′ and other connecting pads are capable of minimizing dispersion of light.

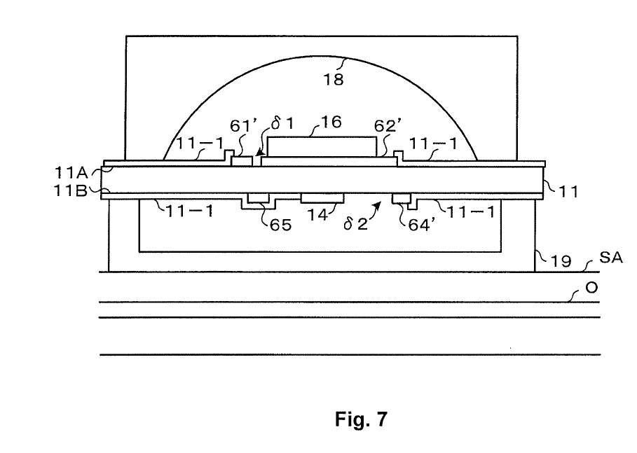

FIG. 7 shows another example of a layout of the light transmission film and the wiring. Structures that are identical to those in the configuration examples described above are indicated by the same numerals, and a description of the structures will not be provided. In the example shown in FIG. 3(B), in cross-sectional view, the light transmission film 11-1 on the first surface 11A of the substrate 11 is present between the wiring connecting pad 61′ and the connecting part 62′. However, in the example shown in FIG. 7, a gap δ1 is present between the connecting pad 61′ and the connecting part 62′. Specifically, in the example shown in FIG. 7, an opening part δ1 is present between the connecting pad 61′ and the connecting part 62′, on a side of the first surface 11A of the substrate 11. However, in the example shown in FIG. 7, a false wiring 65 is formed on the second surface 11B of the substrate 11 opposite the opening part δ1. The false wiring 65 is provided to a region where a wiring is inherently unnecessary, but is provided in order to shield the opening part δ1 from light, and as with the connecting pad 61′, forms a light-blocking region. The false wiring 65 may be a floating wiring, which is not connected to other another wiring that is required, but may also be a redundant portion that is connected to another wiring that is required. Therefore, the false wiring 65 deters the light R1′ having the biological information (i.e., the reflected light) from entering the substrate 11. In an instance in which the false wiring 65 is not present, the light R1′ having the biological information (i.e., the reflected light) disperses at a rough surface on the first surface 11A of the substrate 11 (i.e., the opening part δ1). In the example shown in FIG. 7, since the light transmission film 11-1 is present to the left of the connecting pad 61′, the size of the connecting pad 61′ in FIG. 7 can be set to W+ΔW instead of a dimension shown in FIG. 4(C) so as to account for a displacement in one direction only. In the example shown in FIG. 7, providing the opening part δ1 instead of the light transmission film 11-1 shown in FIG. 3(B) (i.e., the light transmission film 11-1 between the connecting pad 61′ and the connecting part 62′) makes it possible to make the connecting pad 61′ adjacent to the opening part δ1 by ΔW smaller than the connecting pad 61′ shown in FIG. 4(C) (i.e., W+2×ΔW), and is therefore beneficial in an instance in which a constraint is present against increasing the size of the connecting pad 61′.

The false wiring 65 is formed on the second surface of the substrate 11, and the connecting pad 64′, the wiring 64, and similar components are also formed on the second surface 11B of the substrate 11. Therefore, the false wiring 65, the connecting pad 64′, and the wiring 64 can be simultaneously formed using, e.g., photolithography, and are formed from, e.g., a copper foil. The false wiring 65 can thus be readily formed.

Also, in the example shown in FIG. 7, an opening part δ2 is present between the connecting pad 64′ and the light-emitting part 14 on a side of the second surface 11B of the substrate 11, and the connecting part 62′ corresponding to the opening part δ2 is formed on the first surface 11A of the substrate 11. However, the connecting part 62′ in FIG. 7 is extended further to the right (in FIG. 7, the direction of the light-receiving part 16 relative to the connecting pad 61′ is defined as the right) compared to the connecting part 62′ in FIG. 3(B). In the example shown in FIG. 7, the light-blocking region is extended by increasing the size of the connecting part 62′, and the extended light-blocking region is positioned opposite the opening part δ2 on the side of the second surface of the substrate 11 between the connecting pad 64′ and the light-emitting part 14. The connecting part 62′ is formed from, e.g., copper foil, and can be readily formed using photolithography.

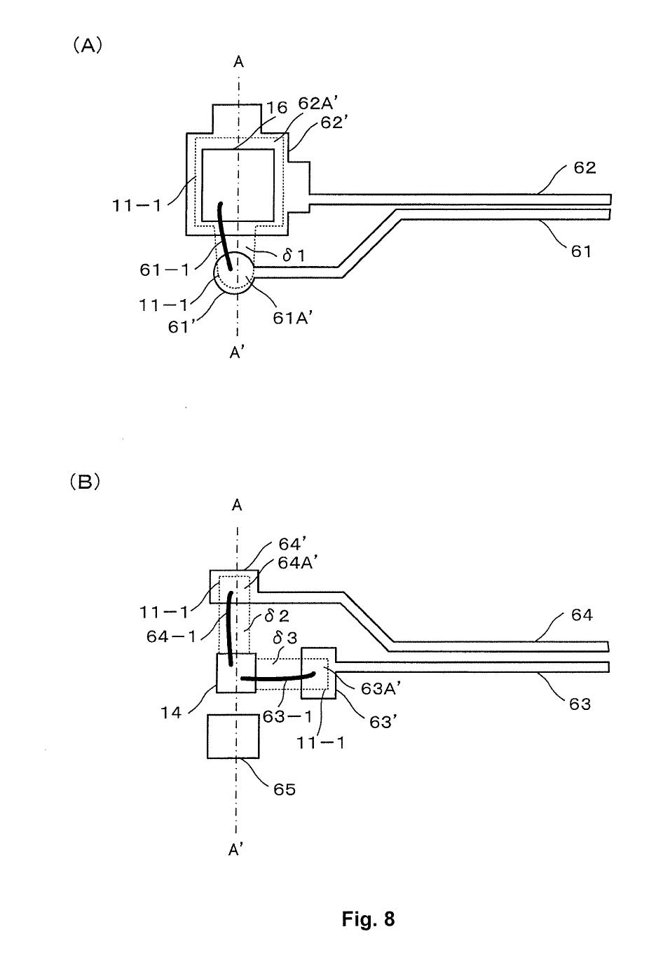

FIGS. 8(A) and 8(B) show another example of a layout surrounding the connecting pad. FIG. 8(A) shows an example of a layout surrounding the connecting pad 61′ in FIG. 7. FIG. 8(B) shows an example of a layout surrounding the connecting pad 64′ shown in FIG. 7. A cross-section view along the line A-A′ in FIGS. 8(A) and 8(B) corresponds to FIG. 7. Structures that are identical to those in the examples described above are indicated by the same numerals, and a description of the structures is not provided.

As shown in FIG. 8(A), the connecting pad 61′ for connecting to the light-receiving part 16 has an exposed part 61A′ in which a part of the surface of the connecting pad 61′ is exposed. Another part of the surface of the connecting pad 61′ (i.e., a part of a periphery) is covered by the light transmission film 11-1. In the example shown in FIG. 8(A), not all of the periphery of the surface of the connecting pad 61′ is covered by the light transmission film 11-1, and an opening part δ1 is therefore formed on the first surface 11A of the substrate 11 between the connecting pad 61′ and the connecting part 62′ (i.e., the light-receiving part 16; see FIG. 7). As shown in FIG. 8(A), with respect to the plan view when viewed from the side towards the light-receiving part 16, the opening part δ1 on the first surface 11A of the substrate 11 is adjacent to the exposed part 61A′ of the connecting pad 61′.

As shown in FIG. 8(B), the connecting pad 64′ for connecting to the light-emitting part 14 has an exposed part 64A′ in which a part of the surface of the connecting pad 64′ is exposed. Another part of the surface of the connecting pad 64′ (i.e., a part of the periphery) is covered by the light transmission film 11-1. In the example shown in FIG. 8(B), not all of the periphery of the surface of the connecting pad 64′ is covered by the light transmission film 11-1, and an opening part δ2 is therefore formed on the second surface of the substrate 11 between the connecting pad 64′ and the light-emitting part 14 (see FIG. 7). As shown in FIG. 8(B), with respect to the plan view when viewed from the side towards the light-emitting part 14, the opening part δ2 on the second surface 11B of the substrate 11 is adjacent to the exposed part 64A′ of the connecting pad 64′.

As shown in FIG. 8(B), a false wiring 65 is formed on the second surface 11B of the substrate 11. The false wiring 65 overlaps with the opening part δ1 on the first surface 11A of the substrate 11 (see FIG. 7) with respect to the plan view. Although the false wiring 65 is not connected to the wiring 63 or another wiring, the wiring 63, the connecting pad 63′, or another wiring may be extended instead of having the false wiring 65.

As shown in FIG. 8(A), the connecting part 62′ on the first surface 11A of the substrate 11 may be extended so as to overlap with the opening part δ2 on the second surface 11B of the substrate 11 (see FIG. 7). A false wiring may be formed on the first surface 11A of the substrate 11 instead of the connecting part 62′ being extended. As shown in FIG. 8(B), the connecting pad 63′ for connecting to the light-emitting part 14 similarly has an exposed part 63A′ in which a part of a surface of the connecting pad 63′ is exposed, and an opening part δ3 is formed on the second surface 11B of the substrate 11 adjacent to the exposed part 63A′. As with the opening part δ2, the opening part δ3 can also be shielded by a wiring or a false wiring on the first surface 11A of the substrate 11.

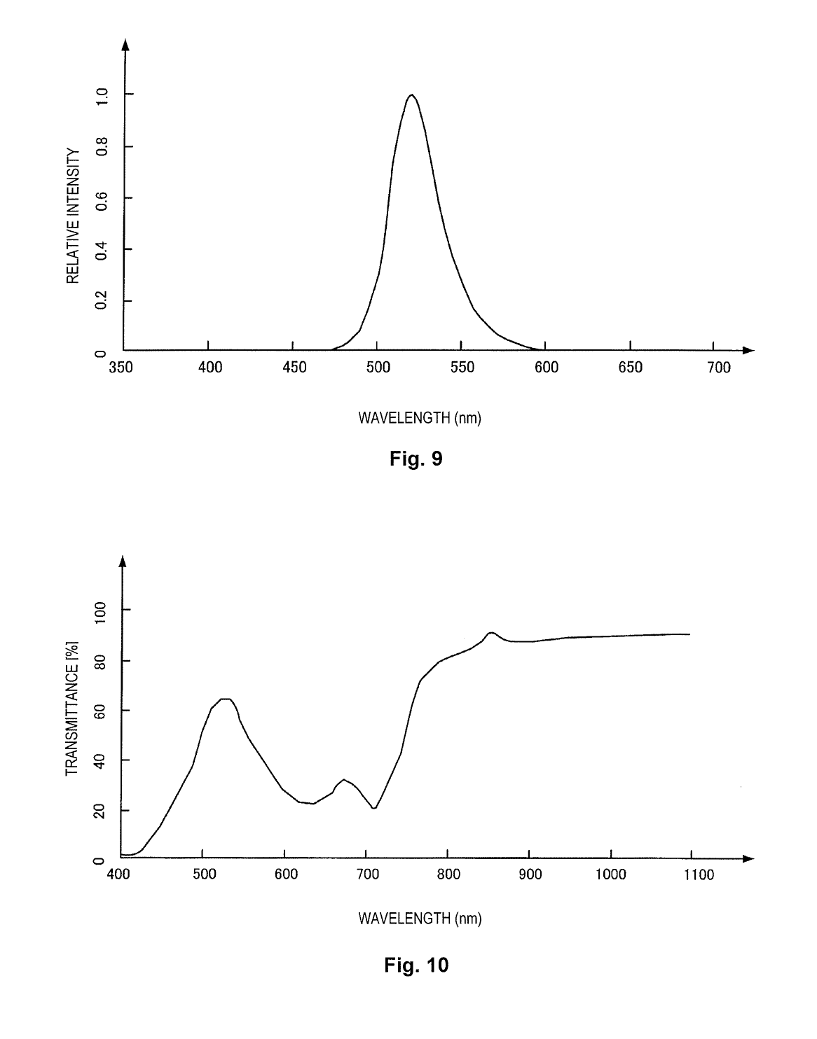

FIG. 9 shows an example of intensity characteristics of the light emitted by the light-emitting part 14. In the example shown in FIG. 9, the intensity is at a maximum for light having a wavelength of 520 nm, and the intensity of light having other wavelengths is normalized with respect thereto. Also, in the example shown in FIG. 9, the wavelengths of light emitted by the light-emitting part 14 are within a range of 470 nm to 600 nm.

FIG. 10 is an example of transmission characteristics of light passing through the substrate 11 coated with the light transmission film 11-1. In the example shown in FIG. 10, transmittance is calculated using the intensity of light before being transmitted through the substrate 11 and the intensity of light after being transmitted through the substrate 11. In the example shown in FIG. 10, in a region of wavelength equal to or less than 700 nm, which is the lower limit of the biological window, the transmittance is at a maximum for light having a wavelength of 525 nm. Or, in the example shown in FIG. 6, in the range of wavelength equal to or less than 700 nm, which is the lower limit of the optical window in biological tissue, the wavelength of the maximum transmittance of light passing through the light transmission film 11-1 falls within a range of ±10% of the wavelength of the maximum intensity of light generated by the light-emitting part 14 in FIG. 9, for example.

It is preferable that the light transmission film 11-1 thus selectively transmit light generated by the light-emitting part 14 (e.g., the reflected light R1′ produced by the first light R1 being reflected in FIG. 1(A), or the first light R1 in FIG. 1(B)). The presence of the light transmission film 11-1 makes it possible to enhance the smoothness of the substrate 11 and prevent, to a certain extent, a decrease in efficiency of the light-emitting part 14 and the light-receiving part 16. In an instance in which transmittance has a maximum value (or in a broader sense, a peak value) within, e.g., a visible light region for light having a wavelength of 525 nm, as shown in the example in FIG. 10, the light transmission film 11-1 is, e.g., green.

FIG. 11 shows another example of a configuration of the biological information detector according to the present embodiment. As shown in FIG. 11, the biological information detector may include a reflecting part 92 for reflecting light, in contrast to the example of a configuration shown in FIG. 7. Structures shown in FIG. 11 that are identical to those in the example described above are indicated by the same numerals, and a description of the structures is not provided. In the example shown in FIG. 11, the light-emitting part 14 generates a first light R1 directed at a detection site O of a test subject (e.g., a user), and a second light R2 directed at a direction other than a direction of the detection site O (i.e., directed at the reflecting part 92). The reflecting part 92 reflects the second light R2 and directs the second light R2 towards the detection site O. The light-receiving part 16 receives light R1′, R2′ (i.e., reflected light), having biological information, the light R1′, R2′ produced by each of the first light R1 and the second light R2 being reflected at the detection site O. The reflecting part 18 reflects the light R1′, R2′ having biological information from the detection site O (i.e. the reflected light) and directs the light R1′, R2′ towards the light-receiving part 16. The presence of the reflecting part 18 causes the second light R2, that does not directly reach the detection site O of the test subject (i.e., the user), to reach the detection site O. In other words, the amount of light reaching the detection site O via the reflecting part 92 increases, and the efficiency of the light-emitting part 14 increases. Therefore, the detection accuracy (i.e., the signal-to-noise ratio) of the biological information detector increases.

In Patent Citation 1, there is disclosed a configuration corresponding to the reflecting part 18 (i.e., a reflecting part 131 in FIG. 16 of Patent Citation 1). Specifically, the light-receiving part 12 in FIG. 16 of Patent Citation 1 receives light reflected at a detection site via the reflecting part 131. However, in Patent Citation 1, a configuration corresponding to the reflecting part 92 is not disclosed. In other words, at the time of filing, those skilled in the art had not been aware of the issue of increasing the efficiency of the light-emitting part 11 in FIG. 16 in Patent Citation 1.

In the example shown in FIG. 11, the false wiring 65 is extended between the reflecting part 92 and the substrate 11. The false wiring 65 is also directly connected to the reflecting part 92 by, e.g., silver paste or another adhesive (not shown). The presence of the false wiring 65 thus makes it possible to readily attach the reflecting part 92 to the substrate 11.

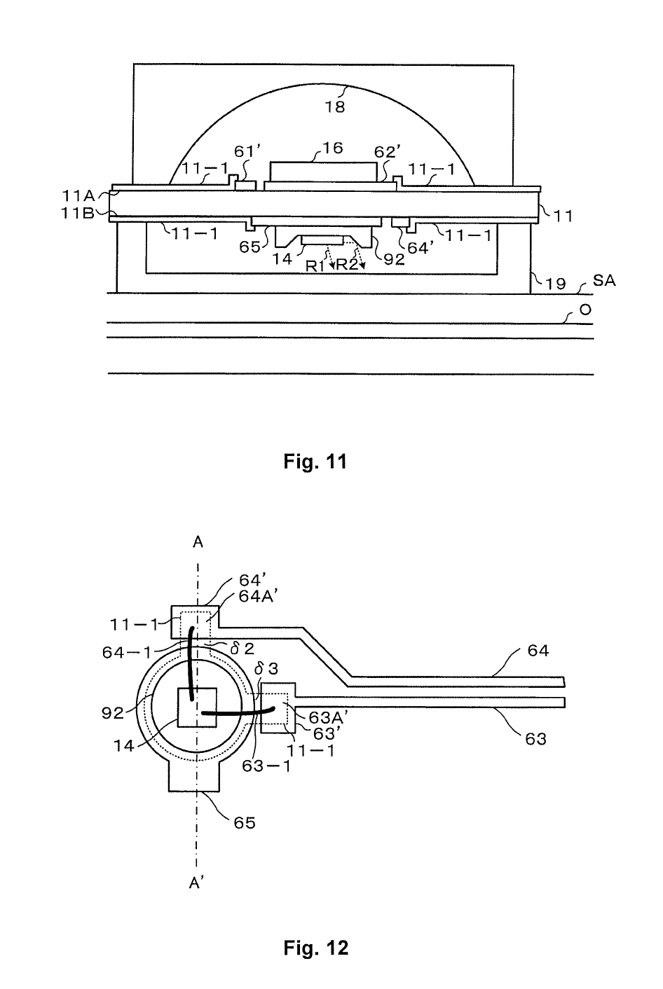

FIG. 12 shows another example of a layout surrounding the connecting pad. FIG. 12 shows an example of a layout surrounding the connecting pad 64′ in FIG. 11. A cross-section view along the line A-A′ in FIG. 12 corresponds to FIG. 11. Structures shown in FIG. 11 that are identical to those in the examples described above are indicated by the same numerals, and a description of the structures is not provided. As shown in FIG. 12, in order to enable the reflecting part 92 to be readily attached to the substrate 11, the area of the false wiring 65 is larger than that of the reflecting part 92 with respect to the plan view. Specifically, with respect to the plan view, the entirety of the reflecting part 92 overlaps the false wiring 65, the reflecting part 92 being located within the area described by the false wiring 65. Also, the false wiring 65 formed on the second surface 11B of the substrate 11 extends to a region that is opposite the opening part δ1 shown in FIG. 8(A), and shields the opening part δ1 from light.

In the example shown in FIG. 12, with respect to the plan view, an outer circumference of the reflecting part 92 is circular, where the diameter of the circle is, e.g., 200 μm to 11,000 μm. The outer circumference of the reflecting part 92 may also be a quadrilateral (or specifically, a square) or another shape with respect to the plan view. Also, in the examples shown in FIGS. 12, the outer circumference of the light-emitting part 14 with respect to the plan view is a quadrilateral (or specifically, a square), where the length of one side of the square is, e.g., 100 μm to 10,000 μm. The outer circumference of the light-emitting part 14 may also be a circle or another shape.

The reflecting part 92 is made of metal whose surface is subjected to mirror surface finishing, and thereby has a reflective structure (or specifically, a mirror reflection structure). The reflecting part 92 may also be formed from, e.g., a resin whose surface is subjected to mirror surface finishing. Specifically, for example, a base metal forming a base of the reflecting part 92 is readied, and a surface of the base metal is then, e.g., subjected to plating. Alternatively, a mold of the reflecting part 92 (not shown) is filled with a thermoplastic resin, molding is performed, and a metal film, for example, is then deposited by vapor deposition on a surface of the mold. The mirror surface part of the reflecting part 92 preferably has a high reflectivity. The reflectivity of the mirror surface part is, e.g., 80% to 90% or higher. In the example shown in FIG. 12, an opening part δ2 is again formed adjacent to the exposed part 64A′ of the connecting pad 64′, and an opening part opening part δ3 is formed adjacent to the exposed part 63A′ of the connecting pad 63′. The opening parts δ2, δ3 can be shielded from light by an extended region of the connecting part 62′ on the first surface 11A of the substrate 11 as shown in FIG. 8(A).

### 2. Biological Information Measuring Device

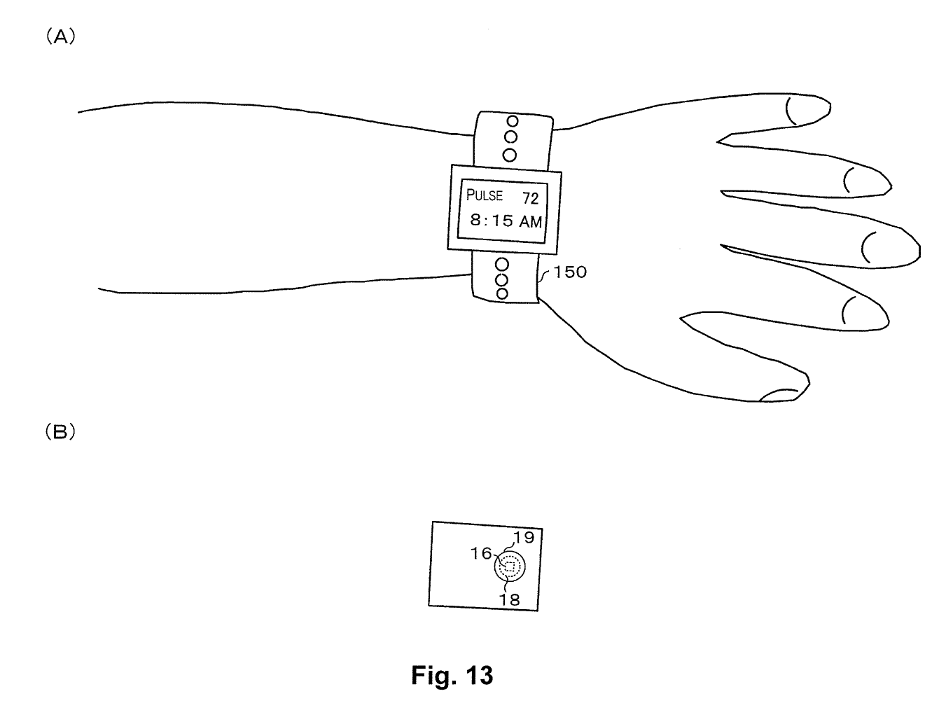

FIGS. 13(A) and 13(B) are examples of the outer appearance of a biological information measuring device including the biological information detector such as that shown in FIG. 1. As shown in FIG. 13(A), the biological information detector shown in, e.g., FIG. 1 may further include a wristband 150 capable of attaching the biological information detector to an arm (or specifically, a wrist) of the test subject (i.e., the user). In the example shown in FIG. 13(A), the biological information is the pulse rate indicated by, e.g., “72.” The biological information detector is installed in a wristwatch showing the time (e.g., “8:15 am”). As shown in FIG. 13(B), an opening part is provided to a back cover of the wristwatch, and the protecting part 19 shown in FIG. 1, for example, is exposed in the opening part. In the example shown in FIG. 13(B), the reflecting part 18 and the light-receiving part 16 are installed in a wristwatch. In the example shown in FIG. 13(B), the reflecting part 92, the light-emitting part 14, the wristband 150, and other components are omitted.

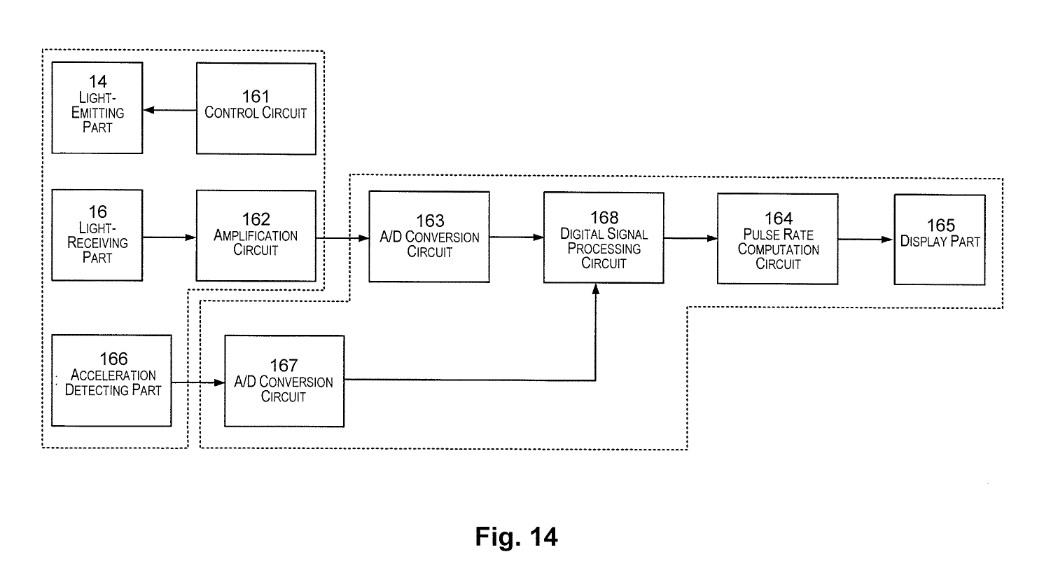

FIG. 14 is an example of a configuration of the biological information measuring device. The biological information measuring device includes the biological information detector as shown, e.g., in FIG. 1, and a biological information measuring part for measuring biological information from a light reception signal generated at the light-receiving part 16 of the biological information detector. As shown in FIG. 14, the biological information detector may have the light-emitting part 14, the light-receiving part 16, and a circuit 161 for controlling the light-emitting part 14. The biological information detector may further have a circuit 162 for amplifying the light reception signal from the light-receiving part 16. The biological information measuring part may have an A/D conversion circuit 163 for performing an A/D conversion of the light reception signal from the light-receiving part 16, and a pulse rate computation circuit 164 for calculating the pulse rate. The biological information measuring part may further have a display part 165 for displaying the pulse rate.

The biological information detector may have an acceleration detecting part 166, and the biological information measuring part may further have an A/D conversion circuit 167 for performing A/D conversion of a light reception signal from the acceleration detecting part 166 and a digital signal processing circuit 168 for processing a digital signal. The configuration of the biological information measuring device is not limited to that shown in FIG. 14. The pulse rate computation circuit 164 in FIG. 14 may be, e.g., an MPU (i.e., a micro processing unit) of an electronic device installed with the biological information detector.

The control circuit 161 in FIG. 14 drives the light-emitting part 14. The control circuit 161 is, e.g., a constant current circuit, delivers a predetermined voltage (e.g., 6 V) to the light-emitting part 14 via a protective resistance, and maintains a current flowing to the light-emitting part 14 at a predetermined value (e.g., 2 mA). The control circuit 161 is capable of driving the light-emitting part 14 in an intermittent manner (e.g., at 128 Hz) in order to reduce consumption current.

The amplification circuit 162 shown in FIG. 14 is capable of removing a DC component from the light reception signal (i.e., an electrical current) generated in the light-receiving part 16, extracting only an AC component, amplifying the AC component, and generating an AC signal. The amplification circuit 162 removes the DC component at or below a predetermined wavelength using, e.g., a high-pass filter, and buffers the AC component using, e.g., an operational amplifier. The light reception signal contains a pulsating component and a body movement component. The amplification circuit 162 and the control circuit 161 are capable of feeding a power supply voltage for operating the light-receiving part 16 at, e.g., reverse bias to the light-receiving part 16. In an instance in which the light-emitting part 14 is intermittently driven, the power supply to the light-receiving part 16 is also intermittently fed, and the AC component is also intermittently amplified. The amplification circuit 162 may also have an amplifier for amplifying the light reception signal at a stage prior to the high-pass filter.

The A/D conversion circuit 163 shown in FIG. 14 converts an AC signal generated in the amplification circuit 162 into a digital signal (i.e., a first digital signal). The acceleration detecting part 166 shown in FIG. 14 calculates, e.g., gravitational acceleration in three axes (i.e., x-axis, y-axis, and z-axis) and generates an acceleration signal. Movement of the body (i.e., the arm), and therefore movement of the biological information measuring device, are reflected in the acceleration signal. The A/D conversion circuit 167 shown in FIG. 14 converts the acceleration signal generated in the acceleration detecting part 166 into a digital signal (i.e., a second digital signal).

The digital signal processing circuit 168 shown in FIG. 14 uses the second digital signal to remove or reduce the body movement component in the first digital signal. The digital signal processing circuit 168 may be formed by, e.g., an FIR filter or another adaptive filter. The digital signal processing circuit 168 inputs the first digital signal and the second digital signal into the adaptive filter and generates a filter output signal in which noise has been removed or reduced.

The pulse rate computation circuit 164 shown in FIG. 14 uses, e.g., fast Fourier transform (or in a broader sense, discrete Fourier transform) to perform a frequency analysis on the filter output signal. The pulse rate computation circuit 164 identifies a frequency that represents a pulsating component based on a result of the frequency analysis, and computationally obtains a pulse rate.

Although a detailed description was made concerning the present embodiment as stated above, persons skilled in the art should be able to easily understand that various modifications are possible without substantially departing from the scope and effects of the invention. Accordingly, all of such examples of modifications are to be included in the scope of the invention. For example, terms stated at least once together with different terms having broader sense or identical sense in the specification or drawings may be replaced with those different terms in any and all locations of the specification or drawings.

What is claimed is:

1. A biological information detector comprising:
a light-emitting part;
a light-receiving part that receives light having biological information, the light being light emitted by the light-emitting part and reflected at a detection site of a test subject;
a reflecting part that reflects the light emitted by the light-emitting part or the light having biological information; and
a substrate having a first surface and a second surface facing the first surface, the light-receiving part being positioned on one of either the first surface or the second surface, and the light-emitting part being positioned on another of either the first surface or the second surface; wherein
the substrate is formed from a material that is transparent with respect to a wavelength of the light emitted by the light-emitting part; and
at least one of either the first surface or the second surface of the substrate has a light-blocking region containing wiring leading to at least one of either the light-emitting part or the light-receiving part, and a light transmission film that is transparent with respect to the wavelength of the light emitted by the light-emitting part, the light transmission film being positioned, with respect to the plan view, at least on a region on the substrate excluding the light-blocking region.

2. The biological information detector according to claim 1, wherein
the wiring has a pad for providing a connection to the light-receiving part, the connecting pad being on the one of either the first surface or second surface,
the substrate has an opening part provided, as viewed from above, adjacent to the connecting pad on the one of either the first surface or the second surface, the light transmission film not being positioned in the opening part, and
the opening part, with respect to the plan view, overlaps with the light-blocking region on the other of either the first surface or the second surface of the substrate.

3. The biological information detector according to claim 1, wherein
the wiring has a pad for providing a connection to the light-emitting part, the connecting pad being on the other of either the first surface or the second surface,
the substrate has an opening part provided, as viewed from above, adjacent to the connecting pad on the other of either the first surface of the second surface, the light transmission film not being positioned in the opening part, and ‘the opening part, with respect to the plan view, overlaps with the light-blocking region on the one of either the first surface or the second surface of the substrate.

4. The biological information detector according to claim 2, further comprising
a false wiring positioned on the light-blocking region overlapping the opening part with respect to the plan view, the light-blocking region being on the other of either the first surface or the second surface of the substrate.

5. The biological information detector according to claim 3, wherein
the wiring has a connecting part in contact with an electrode of the light-receiving part, and
the connecting part is positioned on the light-blocking region overlapping the opening part with respect to the plan view, the light-blocking region being on the one of either the first surface or the second surface of the substrate.

6. The biological information detector according to claim 2, wherein
the connecting pad has an exposed part in which a part of a surface of the connecting pad is exposed,
the opening part is adjacent to the exposed part with respect to the plan view, and
another part of the surface of the connecting pad is covered by the light transmission film.

7. The biological information detector according to claim 1, wherein
the wiring has a pad for providing a connection to at least one of the light-emitting part or the light-receiving part,
the connecting pad has an exposed part in which a part of a surface of the connecting pad is exposed, and
a periphery of the surface of the connecting pad is covered by the light transmission film.

8. A biological information measuring device comprising:
logical information detector according to claim 1; and
a biological information measuring part that measures the biological information from a light reception signal generated in the light-receiving part, wherein
the biological information is a pulse rate.
